(19) United States
(12) Patent Application Publication
(10) Pub. No.: US 2003/0092058 A1
(43) Pub. Date: May 15, 2003

(54) NOVEL FLOW CYTOMETER

(76) Inventor: GLENN F. SPAULDING, HOUSTON, TX (US)

Correspondence Address: THE LAW OFFICES OF COE F. MILES, P.C., 15150 MIDDLEBROOK DRIVE, HOUSTON, TX, 77058, US

(21) Appl. No.: 09/550,276

(22) Filed: Apr. 15, 2000

Related U.S. Application Data
(60) Provisional application No. 60/148,139, filed on Aug. 10, 1999.

(51) Int. Cl. G01N 33/53

(52) U.S. Cl. 435/7.1

(57) ABSTRACT

A novel flow cytometer is described which includes a disposable sealed container that is rotated. Rotation the container directs cells to the inner surface of the container where they are analyzed, and sorted by photoactivated cross-linkers. The flow cytometer is simple, fast and safe.

### FIELD OF THE INVENTION

The present invention relates to a novel flow cytometer, novel methods and new products by processes. A novel container, upon centrifugation, directs and enables the movement of cells, which have been placed in the container, towards the outer walls of the container. A light source and photodetector external to the rotating container interrogates the cells located inside of the container on the outer wall. Said container, light source and photodetector comprise a novel apparatus. Said apparatus and methods of interrogating said cells yield information on cell location, size, shape, cellular constituents, cell volume, and cell buoyancy; and when labeled with a fluorescent or other marker, specific cellular constituents, cell function, and genomic information. Furthermore, the inside surface of the wall can be modified, and/or a modifier can be added to media within the container, such that an external light source can cause the modifiers to selectively immobilize cells to the surface of the wall. Hence, non-immobilized cells would be available for use. Once non-immobilized cells were removed, immobilized cells could be released by chemical, mechanical or photochemical methods. Analysis is accomplished in a sealed disposable container.

### BACKGROUND OF THE INVENTION

Flow cytometry has evolved into a powerful tool for cell analysis and isolation. Standard flow cytometers utilize a complicated system of pressure containers, valves, sheath fluid flows, orienting nozzles and other assorted equipment to move cells in single file through a gas laser light source. All the required fluidics and fluidics processing reduces the optical coupling efficiencies and add instability. To compensate for losses, higher power gas lasers and high sensitivity/high gain photo multipliers are often required; adding complexity, expense and instability when compared to solid state laser diodes and solid state photodetectors. The fluidics, orientating nozzles, large lasers and precision photo multipliers of the present technology, substantially increase cost and reduce reliability. Cell analyses are slow and generally limited by the fundamental physics of fluid flow. During analysis, aerosols are often released; infected cells can impose a substantial risk to the flow cytometer operator.

The novel flow cytometer of this invention alleviates many of the traditional problems associated with flow cytometry by conducting the analysis in a self contained sealed disposable container or cylinder. For example, cells labeled with fluorescent markers are deposited by standard means into a cell guide. The cell guide is contained in a cylinder with a closed end and an open end. The opening can be sealed with a cap or other means. The cylinder is filled with media. As the cylinder is centrifuged, the cell guide directs the cells to the inside of the cylinder wall. A light source and photodetector are positioned outside the cylinder and directed toward the inner cylinder wall where the cells are located. Thus, each cell can be individually analyzed as the cell rotates past the light source and detector. Cylinder rotation provides a novel integrated means that accomplishes cell orientation, cell localization, cell containment within a simple to manufacture and disposable container.

### PRIOR ART

The following are examples of Prior Art, and are herein fully incorporated and with references.

U.S. Pat. No. 5,021,244 issued Jun. 4, 1991 to Spaulding

U.S. Pat. No. 5,346,990 issued Sep. 9, 1994 to Spaulding

U.S. Pat. No. 5,376,267 issued Dec. 12, 1994 to Stokes, et. al.

U.S. Pat. No. 5,439,362 issued Aug. 8, 1995 to Spaulding

U.S. Pat. No. 5,660,997 issued Aug. 8, 1997 to Spaulding

U.S. Pat. No. 6,001,643 issued Dec. 12. 1999 to Spaulding

### SUMMARY OF THE PRESENT INVENTION

The present invention uses a cylindrical container, media and a cell guide to distribute cells over the inner wall of the container or cylinder. This is accomplished by a combination of; an optional cell guide for directing cell distribution, and the balancing of gravitational sedimentation of cells with the centrifuging forces resulting from rotating the container. The cell guide is aligned with the annular axis of the container and located near the opening of the container. Through the center of the cell guide in the annular axis is a passage that is contiguous with the media in the cylinder. Said passage is narrow at the container opening and becomes wider as the passage leads into the container. Cells are deposited at the narrow end of the passage in the cell guide. During centrifugation, and as the cells are sedimenting, the cells are restricted from depositing on the container wall by the cell guide. When the cells sediment beyond the edge of the cell guide, centrifugal forces move the cells toward the inner surface of the wall at the instant when the cell guide no longer restricts lateral movement. Consequently, the release point is defined by the edge and shape of the cell guide passage, therein directing cell location on the inner surface of the wall. Additionally, the centrifugal and sedimentation forces are balanced to further confine cell localization to the wall of the container.

A diode laser and photodiode with integrated amplifier are fixed to a linear actuator aligned with the vertical axis of the container. As the container rotates through 360 degrees, the entire inside wall of the container is scanned at the level of the laser/photodetector. After one level is scanned, the linear actuator moves the laser/photodetector to the next level and scans the container as it rotates 360 degrees. Incrementally scanning each level yields information on cells that have been distributed along the inner surface of the wall. Alternatively, instead of moving the light source and detector, the rotating cylinder is vertically raised and lowered to scan the walls for the rotating cylinder. The information can be useful in determining cell type and proportion. Said information is essential to clinical diagnosis, research and bio/pharmaceutical manufacturing.

To sort cells, the inner surface of the container wall can be modified to include a photoactivated cross-linker. Photoactivated cross-linkers can be activated by the same laser used to analyze the cellular content—using laser energies above those required for analysis. Alternatively, a second laser at a different wavelength, could be used to active the cross-linker. In an alternative embodiment, the cross-linker could be added to the media then photoactivated. To sort a cell, the cell would be analyzed by the laser/photodetector and, based on operator defined parameters, activate the cross-linker or not if the cell did not meet specifications. Those cells that were not cross-linked to the wall could be removed. Those cells that were cross-linked could be released by chemical, mechanical or photochemical means at a latter time.

Flow cytometers that utilize a fluid stream to align cells in single file past a laser/photodetector are limited to the speed of the fluid flow. The addition of multiple lasers/photodetectors to increase throughput would pose substantial challenges because of stream instabilities. The present invention can accommodate many laser/photodetector combinations around the periphery of the rotating container. Laser and CCD array combinations could achieve analysis and sorting rates in excess of 1,000,000 cells/second utilizing current technology, and analyze many more parameters per cells.

It will be appreciated by those of ordinary skill in the art that the present invention is both novel and represents a low cost apparatus and means for analyzing and sorting cells.

### DESCRIPTION OF THE DRAWINGS

In the drawing:

FIG. 1 is a schematized cross sectional view of the cylinder, vertical rotating means, linear actuator means and laser/photodetector.

### DESCRIPTION OF THE PREFERRED EMBODIMENT

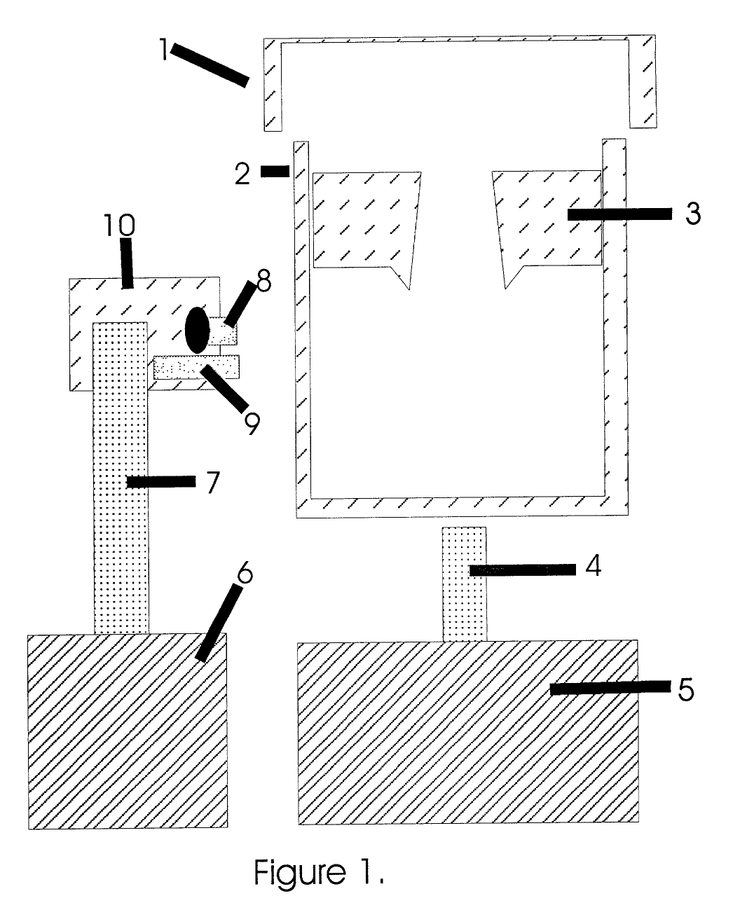

Referring now to FIG. 1, where the schematized cross sectional view shows the general organization. The cylinder or container having an open end and a closed end 2 has a cell guide 3 inserted into the cylinder. The cell guide has a passage through it, and is disposed in a manner such that the smaller end of the passage faces the cap 1 or other means to seal the cylinder. The larger end of the passage through the cell guide 3 opens into the cylinder 2, both are filled with media during the process of centrifugation. The cylinder 2 is vertically rotated by a motor means 5 having a shaft 4 disposed to the cylinder in a fashion that would allow rotation around the vertical axis of cylinder 2. A light source 9 such as an LED or laser and a photodetector 8 are adapted to interrogate cells that are dispersed to the inner surface of the wall of cylinder 2 during centrifugation. The light source 9 and photodetector 8 are disposed to a linear motion means for vertical up/down movement 6 with shaft 7 by means 10; means 10 is adapted to precisely position the light source 9/photodetector 8 with respect to the cylinder 2. Said linear motion means 6 and shaft 7 is used move to the light source 9/photodetector 8 along the vertical axis of cylinder 2. Alternatively, the linear motion means are adapted to vertically move said rotating cylinder while the photodetectors and light sources remain in a fixed position.

### EXAMPLE 1

A 400 nm laser diode and photodiode with integrated amplifier was mounted to a linear actuator. A 1.3 inch I.D. by 1.7 inch tall cylinder with cap was mounted on a stepper motor and microstepped for vertical rotation. Light was collected from the inner wall of the rotating cylinder through a 500 nm long pass filter to the photodiode/amplifier. The amplified voltage signal was sampled by a 12 bit ADC at each microstep and plotted. After the cylinder was scanned and plotted for 360 degrees of rotation, the linear actuator was stepped to the next level. The cylinder was again scanned and plotted through 360 degrees. Once each level in the vertical direction was collected the data was analyzed.

### EXAMPLE 2

The inner wall of the cylinder is modified for photo cross-linking by SurModics, Inc. Eden Prairie, Minn. or an organic photoreceptor material optimized for a wave length from 300 nm to 2000 nm which could include dibromo anthanthrone, titanyl pthalocyanine or other suitable receptors for cross-linking. When a threshold for a parameter is met, the laser power is increased to induce photo cross-linking of the cell to the wall. Alternatively, when a threshold for a parameter is met, a second laser at a different wave length and a known microstep distant away, is utilized to activate the cross-linker.

### EXAMPLE 3

A blue LED or blue laser diode is modulated and synchronously demodulated as the light is scanned across the cylinder to remove ambient from the signal, i.e., by subtracting background.

### EXAMPLE 4

Rare event analysis can be accomplished by reanalyzing cells. Cells are analyzed as previously described. Cells that rare as defined by fluorescent or other markers, are reanalyzed by positioning the laser and photodetector near the rare event and rescanning the area. Alternately. once analyzed new fluorescent probes can be added to the media to label cells which have been previously analyzed, for additional information or for conformation purposes.

### EXAMPLE 5

Biomass analysis can be accomplished by counting the number of cells, determining size of each cell based on light scatter, and integrating the two parameters to determine biomass.

### EXAMPLE 6

Confocal analysis can be accomplished by 360 degree scanning at each vertical level as previously described, then stepping the laser/photodetector in the horizontal axis for each complete scan, i.e. sectioning each cell in the horizontal plane. By modifying the optics to confocal optics, as is known in the art, and stepping the scan in the horizontal plane, the cells will be section into vertical slices that can be digitally reassembled to provide a 3-dimensional model/image of each cell.

### EXAMPLE 7

For improved quality control and to track samples, a bar code is disposed to the container. Since the container or cylinder is vertically rotated, a bar code reader placed in the cytometer apparatus of FIG. 1 could read the bar code as the container is rotated.

### EXAMPLE 8

In an alternate embodiment, the optical components, essentially consisting of the photodetector, an excitation source, and appropriate optical filters, were fixed in place to preclude movement. In this alternate embodiment, the rotating cylinder was moved up and down by a motor means, e.g.; linear actuator or worm gear assembly or gear means.

### EXAMPLE 9

The rotating cylinder presents a unique optical model; both inner and outer surfaces are curved. Curved surfaces result in optical paths that require smaller optics to efficiently accommodate analysis on the inner surface of the cylinder. Conventional flat surface light excitation and collection is better served by increasing the collection area, i.e., conventional photomultiplier tubes, large area avalanche photodiodes. In the instant invention, an EG&G Channel Photomultiplier having a photocathode collecting area of only 5 mm was utilized. The unique combination of curved inner and outer cylinder surfaces with a channel photomultiplier having a small collection area, low noise and high gain exceeding 10ˆ 8 yielded unexpected improvements in fluorescent sensitivities. Moreover, a channel photomultiplier configured in photon counting mode further improved sensitivity and detection.

### EXAMPLE 10

To enhance instrument to instrument standardization and calibration, a calibration standard was designed. Said standard consisted of calibrated standards including but not limited to; beads, fluorescent beads, DNA, RNA, chemical compounds, and such, affixed to the inner cylinder wall. Said cylinder with affixed standards, herein referred as calibrating cylinder, was placed in the spinning cytometer of this invention, and raster scanned as previously described. The data obtained from the calibrating cylinder and the calibrating cylinder can then be utilized as a standard by other spinning cytometers. The instant invention being the cylinder with standards affixed to the inner wall which is vertically rotated and scanned.

### EXAMPLE 11

In an embodiment for maintaining optical focus at a particular distance from the inner cylinder wall, the light source/collection lens assemblies were mounted on coil in a magnetic field. This approach is known in the art and is commonly used to focus a laser beam and collect light from a computer disk or CD. In a CD player, 2 diffraction bands generated from a diffraction grid and laser beam are focused on the flat CD surface. Changes in CD distance and track location can be deduced by comparing the two side diffraction beams. In the instant invention, the diffraction gradient is eliminated instead utilizing the unique optical properties of the rotating cylinder. A unique property of the spinning cytometer is the curved surface comprised of an inner wall surface and an exterior wall surface. Both surfaces reflect light and create fringe patterns with specific peak to trough, and peak intensity differences that are proportional to the distance the cylinder is to the laser light source and proportional to the thickness of the cylinder wall. By placing one or more photodetector or a CCD array in the light path of a fringe pattern (peak and/or trough), the detector signal is used to dynamically compensated focusing through closed-loop feedback to the focusing assembly. Integrated circuits for feedback focusing can be purchased individually or as an assembly with the focusing optics, as is known in the art.

### EXAMPLE 12

In an embodiment for a simple diagnostic test, the media in the cylinder contains labeled antibodies, labeled DNA, labeled RNA, labeled protein, labeled beads, labeled magnetic beads, radioactive markers, chemically modified beads, probes or other such markers (collectively known as treated or treatment) that are listed and can be obtained from for example Molecular Probes, Eugene, Oreg., that bind to cells, cellular constituents, DNA, RNA, bacteria, virus, fungi; collectively referred to as organic matter. When a cell or other product is placed in the media, one or more of the above list labels, markers, or probes binds to constituent of interest during centrifugation to the inner cylinder wall. For example, a heterogeneous population of cells is placed in the cylinder containing media. The media contains a florescent labeled antibody to a specific subppulation of the heterogeneous population. As all cell types are centrifuged through the media containing antibodies, the antibodies binds to the targeted subpopulation—and are concentrated on that targeted cell. After a suitable time for all the cells to be centrifuged to the inner cylinder wall, the wall is raster scanned for fluorescent emissions; as previously described. Those cells with labeled antibody bound to them will fluoresce above background, due to the concentrating effect, and be distinguishable from those cells without antibody labeling. The number of unlabeled cells, and other parameters, can be obtained by collecting laser light scatter. This novel combination of processing and analysis in the same spinning cytometer constitutes a novel diagnostic test. Said testing may be for; gene expression, DNA or RNA sequence identification, hematological testing, cancer screening, cell phenotype, drug screening, AIDS screening, screening for infectious materials, screening for expression of green fluorescent protein, hybridization studies, metabolic screening, scintillation studies, growth studies, luminescence, pap screening, size and shape determinations, biomass determination and the like.

### EXAMPLE 13

In an embodiment for disaggregating cells for analysis, the media inside of the cylinder contains an enzyme for disaggregating cells. Said enzymes are known in the art and include collagenase, dispase, and trypsin. Aggregated cell are added to the media containing enzyme and centrifuged for analysis as previously described. In an alternate embodiment, the cylinder containing media, cell aggregates, and enzyme, is rotated in alternating directions to enhance desegregation; prior to analysis.

### EXAMPLE 14

In an alternate embodiment for analysis, said cell guide was removed and cells and bacteria were suspended by agitation in the media. Once suspended, the cylinder containing media and cells and bacteria were rotated for analysis as previously described.

### EXAMPLE 15

In an embodiment for multi-parameter collection capabilities, different sensor are positioned to surround the rotating cylinder. Said sensor can include one or more of the following sensors, CCD arrays, light scatter detectors, multi-color detectors, infrared detectors, photon counters, scintillation detectors, radioactivity detectors, Confocal microscopy collection optics, and the like. Various light sources can be positioned to surround the rotating cylinder and work in concert with a combination of sensors. Said light sources can include ultraviolet LEDs, visible LEDs, infrared LEDs, ultraviolet diode lasers, visible diode lasers, infrared diode lasers, gas lasers, incandescent sources, and the like. For example, a 480 nm or 400 nm laser diode and photodetector is positioned at 0 degrees, a scintillation detector is positioned at 90 degrees, and a Confocal objective and camera assembly is positioned at 180 degrees. A tritiated cell population labeled with a fluorescent antibody tag is added to the media, and the cylinder centrifuged until the cells are distributed along the inner wall. As a particular cell is analyzed by the 0 degree detector, the fluorescent intensity and position is stored. The same cell is then rotated through the 90 degree detector and the amount of radioactivity measured and stored along with position. The same cell is then rotated through the 180 degree detector and a photomicrograph taken and stored along with its position. Since the position of the detector and cylinder are known, a data base of different sensor data can be collected for each cell. As such, the two dimensional coordinate system (up/down and rotation angle) becomes the data base header; data collected from each detector is stored in the position bin. Moreover, if a cell requires reanalysis at a higher gain the cell can be rotated back to a specific detector, the gain can be increased and the data collected with improved sensitivity.

### EXAMPLE 16

In an alternate embody for cylinder design, the wall of the cylinder was sloped. The diameter of the bottom of the cylinder was narrower than the top. A negative slope resulted in two improvements; the cylinder was easer to remove from the injection molding die, and the negative slope partially counter balanced the sedimentation forces affecting the cells after they were centrifuged on to the inner wall.

### EXAMPLE 17

In an embodiment for selecting X and Y sperm, the sperm DNA is labeled with a fluorescent label, for example H33342 or a sex chromosome specific probe. The mixed sperm population is added to a cylinder in which the inner wall is coated with a photoactivated cross-linker. The population with the undesired fluorescent intensity is cross-linked by photoactivation to the inner cylinder wall. The population with the desired fluorescent intensity therein remains mobile and can be removed by pouring off the media. In an alternate embodiment, the subpopulation with the undesired fluorescent intensity is exposed to an extreme laser intensity that damages or kills the cell. This method and the product by process can be applied to the isolation of stem cells and other specific subpopulations.

### EXAMPLE 18

In an embodiment for disposing the cylinder to a vertical rotating means, a conical indentation is molded into the closed end of the cylinder for cylinder alignment. Said indentation is aligned with the annular axis of said cylinder. Tabs are molded into the closed end of said cylinder, and utilized to mechanically lock said cylinder to a rotating means. Said rotating means has a conical extension that fits into said conical indentation so to stabilize and precisely align said rotating cylinder. Said combination of tabs for mechanical tightening down said matching conical indentation to said conical extension forms a unique mechanism for disposing said cylinder to said rotating means; and holds and precisely aligns said cylinder for raster scanning.

I claim:

1. A cytometer apparatus comprising:
a vertical rotating means adapted to receive and vertically rotate a transparent cylinder,
a light source and detector adapted to interrogate the wall of said vertically rotating transparent cylinder, and
said vertically rotating transparent cylinder or light source and detector moved vertically to interrogate the wall of said vertically rotating transparent cylinder.

2. The transparent rotating cylinder as set forth in claim 1 having a bare code label affixed to its outer wall,
said bar code label vertically aligned, and
said bar code label interrogated by said light source and detector.

3. The transparent rotating cylinder as set forth in claim 1 having calibration standards affixed to its inner wall, and
said affixed standards interrogated by said light source and detector.

4. The transparent rotating cylinder as set forth in claim 1 having a photoactivated cross-linker on its inner wall.

5. A cell guide for the centrifugal dispersion of cells comprising:
a cell guide member inside a vertically rotating transparent cylinder,
said cylinder having a closed end and an open end,
said cell guide member having a passage through its annular axis,
said passage being narrower at one end, and
said narrow end disposed facing the cylinder opening.

6. A diagnostic method comprising;
disposing treated organic matter in a transparent cylinder,
said transparent cylinder vertically rotated through an interrogating light source and detector,
said detector detecting signals from said treated organic matter, and
said signal being diagnostic.

7. A method for sorting cells comprising;
disposing treated cells in a transparent cylinder,
said cylinder having a photoactivated cross-linker disposed to its inner wall,
said transparent cylinder vertically rotated through a light source and detector,
said detector detecting a signal from said treated cells,
said signal achieving a threshold and activating a light source, and
said activating light source activating said photoactivated cross-linker and linking said treated cells to said cylinder.

8. The subpopulation of cells selected from a larger population of cells by the method of claim 7 wherein said subpopulation was not linked to said cylinder wall.

9. The subpopulation of cells selected from a larger population of cells by the method of claim 7 wherein said subpopulation was linked to said cylinder wall.
